(19) United States
(12) Patent Application Publication
(10) Pub. No.: US 2007/0088386 A1
(43) Pub. Date: Apr. 19, 2007

(54) Apparatus and method for treatment of soft tissue injuries

(76) Inventor: Eilaz P. Babaev, Minnetonka, MN (US)

Correspondence Address: CYR & ASSOCIATES, P.A., PONDVIEW PLAZA, 5850 OPUS PARKWAY SUITE 114, MINNETONKA, MN, 55343, US

(21) Appl. No.: 11/252,680

(22) Filed: Oct. 18, 2005

(51) Int. Cl. A61B 17/00

(52) U.S. Cl. 606/204

(57) ABSTRACT

The present invention is a multi-purpose method and device comprising a therapeutic body containing a plurality of pins. The distal ends of the pins on the therapeutic body are placed in contact with the skin. The apparatus may be used to provide treatment to persons and animals suffering from the symptoms of soft tissue injuries in various regions of the body. The device and method is also useful for relaxation and reenergizing of an injured or tired body.

### BACKGROUND OF THE INVENTION

1. Field of the Invention

The present invention relates to orthopedic devices and, more particularly, to orthopedic devices and methods that utilize a plurality of discrete pressure points.

2. Description of the Related Art

The usual treatment for relieving pain from a typical case of muscle and soft tissue injury involves more than merely applying both cold and/or heat packs, resting the injured area, and alleviating any associated pain. Often an external device such as a bandage is used to support and reduce the pain of injured muscles and soft tissues during or before normal activities.

Existing devices and methods for relieving pain from soft tissue injury have been directed to lateral epicondylitis due to inflammation of the extensor muscle mass. Such devices include elastic and non-elastic tension bandages which may, for example, be fastened around the forearm for support and to inhibit the massive movement of the extensor and flexor muscles and to absorb shock. One form of bandage for alleviating the symptoms of tennis elbow relies upon an inflatable bladder for localizing arm pressure. Similar bandages may incorporate a pocket that holds a hot or cold pack. The pressure point of the bandage may be adjustable. Some bandages direct mechanical force to a particular unitary point.

The aforementioned devices typically support and inhibit the massive movement of extensor and flexor muscles, absorb shock, and may include thermal features. Although producing some benefits, current devices do not provide directed pressure simultaneously in multiple points and simultaneously in different directions such as radial, longitudinal and superficial. Moreover, conventional bandages typically do not have immediate or long-term pain relief or relaxation effects.

Chinese acupuncture, which is well known and recognized worldwide, is also used for treating soft tissue damage. Acupuncture is the method of inserting of needles into to skin at exact defined specific Bioactive Points (BAP) in order to treat different disorders, give pain relief, achieve relaxation, and attain other beneficial effects known to those skilled in the art. Acupuncture requires a skilled and specially trained, even certified, practitioner. Sterile needles, clinical space, and treatment time for a patient to be under needles are also required. In addition, there is a danger of infection when the skin is punctured.

Acupressure is the application of pressure at the traditional acupuncture points. Acupressure is similar to acupuncture but does not require insertion of needles into the skin so that acupressure is safe from infection-related side effects. However, acupressure still requires a skilled and specially trained practitioner because the localized high mechanical pressure being applied to one point is sometimes painful.

### SUMMARY OF THE INVENTION

The present invention is directed toward apparatus and methods for the relief of pain and promotion of healing of damaged soft tissues. Apparatus and methods in accordance with the present invention may meet the needs described above and may also provide additional advantages and improvements that will be recognized by those skilled in the art upon review of the present disclosure. The present invention may provide a band adapted to be worn by persons and animals suffering from the symptoms of muscle or soft tissue injuries that result in muscle and soft tissue pain such as tennis elbow carpal syndrome. Apparatus and methods in accordance with the present invention may be useful for the relaxation of injured or tired muscles and soft tissues of the body. Apparatus and methods in accordance with the present invention may be used by orthopedic patients, athletes, physically active people, and animals. Upon review and understanding of the present disclosure, those skilled in the art may recognize modifications of the apparatus for use on various parts of human or animal anatomy without departing from the scope of the present invention as set forth in the claims.

The apparatus of the present invention provides a therapeutic body having a plurality of pins protruding distally from a therapeutic body. The plurality of pins may concentrate pressure at multiple locations on the surface of the over a defined region of the body that is being treated. The apparatus will typically be positioned with the therapeutic body in contact with the skin proximate to the injured soft tissue. The pins are defined as ordinary pins or other protrusions capable of providing pressure at a region localized such that a therapeutic benefit is conferred. The pins apply generally directed pressure, radially and longitudinally, to the area being treated and the pins may act to absorb shock.

The apparatus of the present invention may avoid the problems associated with acupuncture and with acupressure by using a plurality of pins affixed to a therapeutic body to apply pressure to the skin surface without breaking the skin surface. The density of the pins on the therapeutic body may be as great as approximately 1,000 pins per cm². More typically, the density of the pins on the therapeutic body lies between 10 pins per cm² and 200 pins per cm² with a suggested density of 100 pins per Cm². Use of a plurality of pins will typically distribute the force applied to the skin surface by each pin thereby preventing insertion of pins into the skin and the concomitant risk of infection as well as the pain associated with the application of pressure at a single point.

Multiple pins may stimulate the dendrites of each skin area in contact with a pin. The increased contact area from the use of multiple pins may increase the therapeutic effects within the body. The skin adjacent to the treated soft tissues is uneven so that the multiple pins are able to contact the skin surface at varying orientations with respect to the skin surface which stretches the skin surface so as to open pores on the skin surface which increases the absorption of oxygen through the pores. Increasing the pin density may distribute the force to cause stimulate more nerves and cause more skin stretching with a corresponding increase in oxygen absorption. Use of multiple pins may also avoid the need for locating specific BAP.

The therapeutic body may include a band wherein the band is adapted to be worn by persons suffering from the symptoms of muscle or soft tissue injuries such that the therapeutic body is applied to the skin surface proximate to the injured soft tissue. The force with which the therapeutic body contacts the skin surface may be adjusted by adjusting the tension of the band about the body.

The pins may be composed of metal, plastic, ceramic, stone, rubber, wood, fabric, or combinations thereof, or other materials alone or in combination as would be recognized by those skilled in the art.

The pins may have pointed, cylindrical, conical, spherical, or curvilinear shapes. The different pin shapes are useful for different therapeutic purposes. For example, the pointed shape is effective before and after physical activities and is suggested for use for pain relief, for alleviating discomfort, during relaxation, and before sleeping. The spherical or curvilinear shapes are suitable for effecting the strengthening of the muscles in training, and are useful before, during and after physical activities and during the healing process. The cylindrical and conical shapes are recommended for pain relief, low physical activities, and alleviating discomfort.

In addition, the apparatus may include additional elements such as a bladder containing air or a liquid including a gel and may also include devices that regulate temperature. The apparatus may contain elements that introduce medications. Stimulators like electrodes, sonotrodes, and diodes may also be included.

The therapeutic body may be rigid or soft or combinations of rigid and soft. The pins may be arranged in various configurations upon the therapeutic body.

Various embodiments of the apparatus may be used for treatment of different regions of the body having orthopedic, traumatic, pain related problems. Use of the apparatus may provide relaxation of tired or injured body parts and the alleviation of the pain of muscles and soft tissues. The use of localized pressure at multiple locations may provide a number of advantages such as absorption of shock, supporting and reducing the pain of injured muscles and soft tissues before, during or after normal activities, reducing the risk of further injury, allowing the patient to feel comfortable, and giving the damaged area time to heal, creating multiple islands in between injured parts for healing, and accumulating energy before physical activities. The apparatus of the present invention may reduce risk of further injury, allows patient to feel comfortable and gives time for the damaged area to heal. These and other novel aspects of the invention will become more apparent from the discussion and drawings below.

### BRIEF DESCRIPTION OF THE DRAWINGS

FIG. 1 illustrates a perspective views of an embodiment of the present inventions showing a therapeutic body attached to a strap;

FIG. 2 illustrates bottom views of various embodiments for geometric arrangements of pins on a therapeutic body in accordance with the present inventions;

FIG. 3 illustrates a side view and bottom view of an embodiment of a therapeutic body having pins with rounded distal ends in accordance with the present inventions;

FIG. 4 illustrates a side view and bottom view of an embodiment of a therapeutic body containing pins having pointed distal ends in accordance with the present inventions;

FIG. 5 illustrates a side view and bottom view of an embodiment of a compliant therapeutic body containing a plurality of pins in accordance with the present inventions;

FIG. 6 illustrates a side view of an embodiment of a compliant therapeutic body including a bladder in accordance with the present inventions;

FIG. 7 illustrates a side view and bottom view of an embodiment of an apparatus in accordance with the present inventions having a segmented therapeutic body;

FIG. 8 illustrates a side view and bottom view of an embodiment of an apparatus in accordance with the present inventions having a segmented therapeutic body;

FIG. 9 illustrates various target locations on a body for therapeutic treatment by apparatus in accordance with the present inventions;

FIG. 10 illustrates an embodiment of an apparatus in accordance with the present inventions wherein the therapeutic body is formed as a blanket; and

FIG. 11 illustrates an embodiment of the resultant mechanical forces within the skin conferred by contact between the distal end of a pin and the skin surface of an apparatus in accordance with the present inventions.

### DETAILED DESCRIPTION OF THE INVENTION

The present inventions provide for treatment of persons and animals suffering from symptoms of soft tissue injuries such as tennis elbow, carpal syndrome, and muscle pains and may also relax of an injured or tired muscle in various parts of the body such as the head, forehead, ear, face, neck, chest, arm, hand, wrist, back, abdominal, legs, knee, and foot.

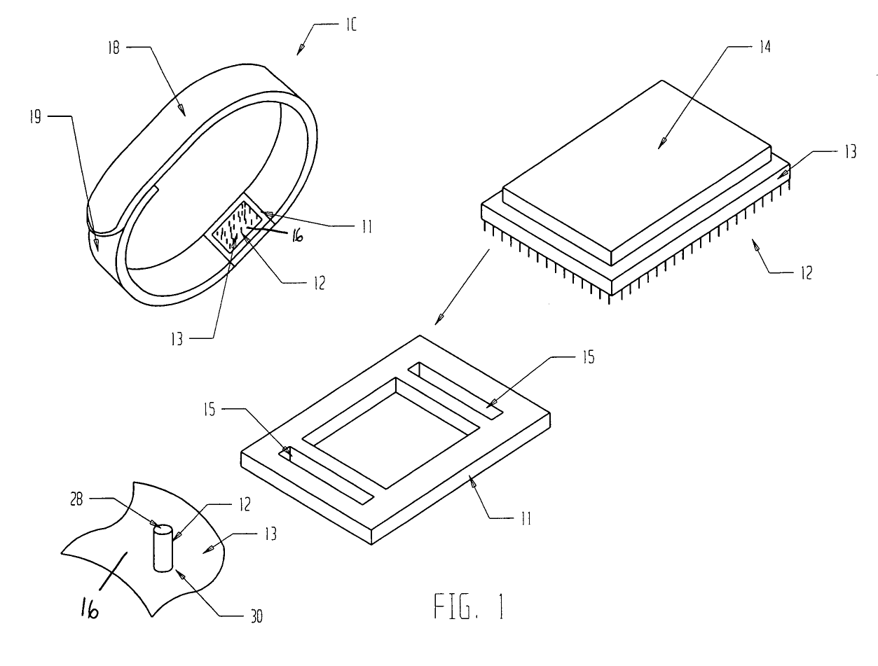

FIG. 1 generally illustrates an apparatus 10 including embodiments of aspects in accordance with the present inventions. The apparatus 10 of the present invention includes a therapeutic body 13 having a therapeutic surface 16. The therapeutic body 13 includes a plurality of pins 12 secured to the therapeutic body 13. The pins 12 extend from a therapeutic surface 16 of the therapeutic body 13. The therapeutic surface 16 may be planar or undulating and may be generally shaped to conform to a skin surface against which the therapeutic surface 16 is to be placed. The pins 12 may be ordinary pins or otherwise configured as protrusions capable of providing pressure at a region localized such that a therapeutic benefit is conferred. Each pin 12 has two ends, a distal end 28 and a proximal end 30, also shown in FIG. 1. The pins 12 protrude distally from the therapeutic surface 16 of the therapeutic body 13 and may protrude substantially normal to a therapeutic surface 16 of the therapeutic body 13. The proximal end 30 is typically secured to or within the therapeutic body 13. The distal end 28 extends from the therapeutic surface 16 to produce pressure concentration 22 on the skin surface 20, as shown in FIG. 11, when the therapeutic body 13 is positioned with the distal ends 28 of the affixed pins 12 in contact with the skin surface 20.

Various mechanisms may be used to position the therapeutic body 13 such that the distal ends 28 of the affixed pins 12 are held in contact with the skin surface. For example, the therapeutic body 13 may be attached to a cartridge 14 where the cartridge 14 is detachably received by a cartridge holder 11, as illustrated in FIG. 1 so that the therapeutic body 13 in the cartridge holder 11 may be replaced.

In the embodiment shown in FIG. 1, a strap 18 is passed through eyes 15 of the cartridge holder 11 and folded back and attached to itself so that the cartridge holder 11 is secured to the strap 18. The cartridge holder 11 may be secured to the strap 18 in other ways as would be recognized by those skilled in the art.

The strap 18 may be securably fastened so as to position the therapeutic body 13 in contact with the skin 20 in different ways, such as by use of Velcro 19, as shown in FIG. 1, or by inserting the clasp of a buckle through holes in the strap, or by the use of other mechanisms readily recognizable to those skilled in the art.

In other embodiments, the therapeutic body 12 may be permanently attached to the strap 18. A plurality of straps 18 may also be used. Other mechanisms may also be used to position the therapeutic body 13 such that the distal ends 28 of the affixed pins 12 are held in contact with the skin surface.

Various embodiments of the therapeutic body 13 and pins 12 may be used based on the type of soft tissue injury and the part of the body being treated. For example, the pins 12 may be arranged on the therapeutic body 13 in various patterns, as shown in FIG. 2. The patterns may be linear, sinusoidal, triangular, rectangular, circular, spiral or combinations thereof as shown in FIG. 2 (a), (b), (c), (d), (e), (f) and (g) respectively.

The pins 12 are defined as ordinary pins or otherwise configured as protrusions capable of providing pressure at a localized region on the skin surface. The region is localized such that a therapeutic benefit is conferred through stretching of the skin and stimulation of the underlying nerves due to the application of radially and longitudinally directed pressure to the skin surface by the pins.

The pins 12 may be made from plastic, metals such as steel, precious metals which would include gold, silver, and platinum, or other materials.

FIG. 3 illustrates an embodiment of a therapeutic body 13 containing pins 12 with rounded distal ends 28, and FIG. 4 illustrates and embodiment of a therapeutic body 13 containing pins 12 with pointed distal ends 28.

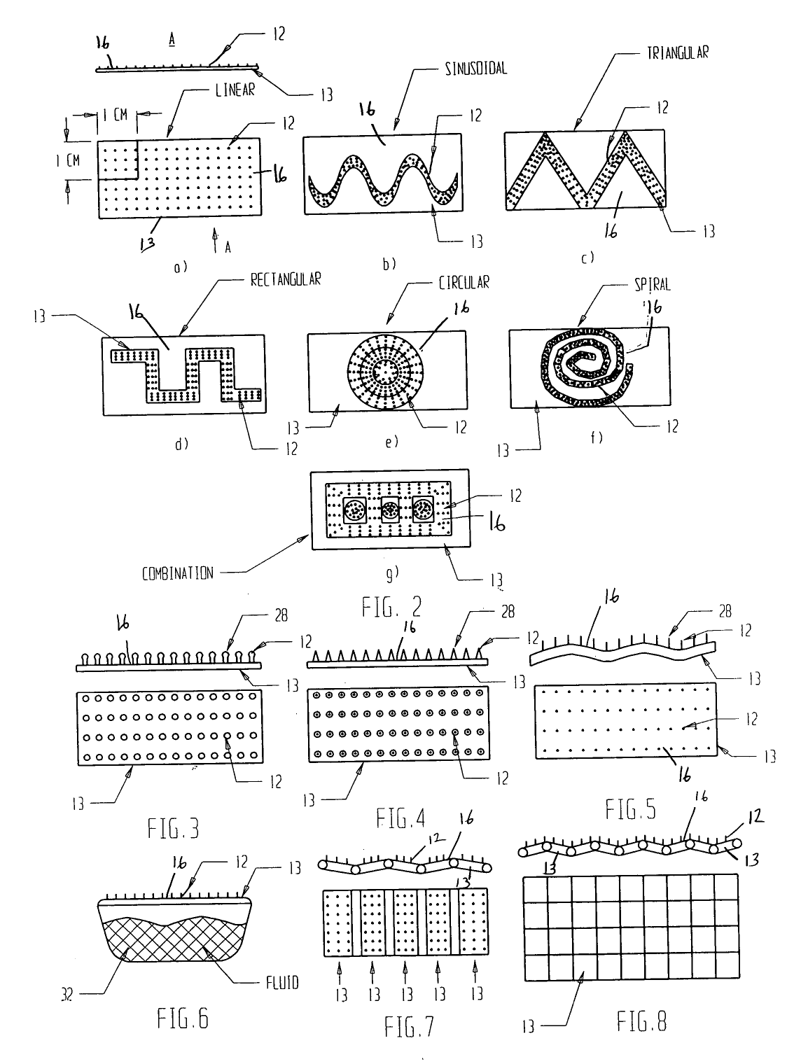

The therapeutic body 13 can be made from a compliant material, as shown in FIG. 5 or from a rigid non-compliant material, as shown in FIGS. 2, 3, and 4. The therapeutic body 13 may be segmented, as shown in FIGS. 7 and 8.

A compliant therapeutic body 13 mounted on a bladder 32 containing a fluid such as air, water, or a gelatinous fluid, is shown in FIG. 6. Hot or cold fluids may be used and various embodiments may include features that control the temperature of the fluid.

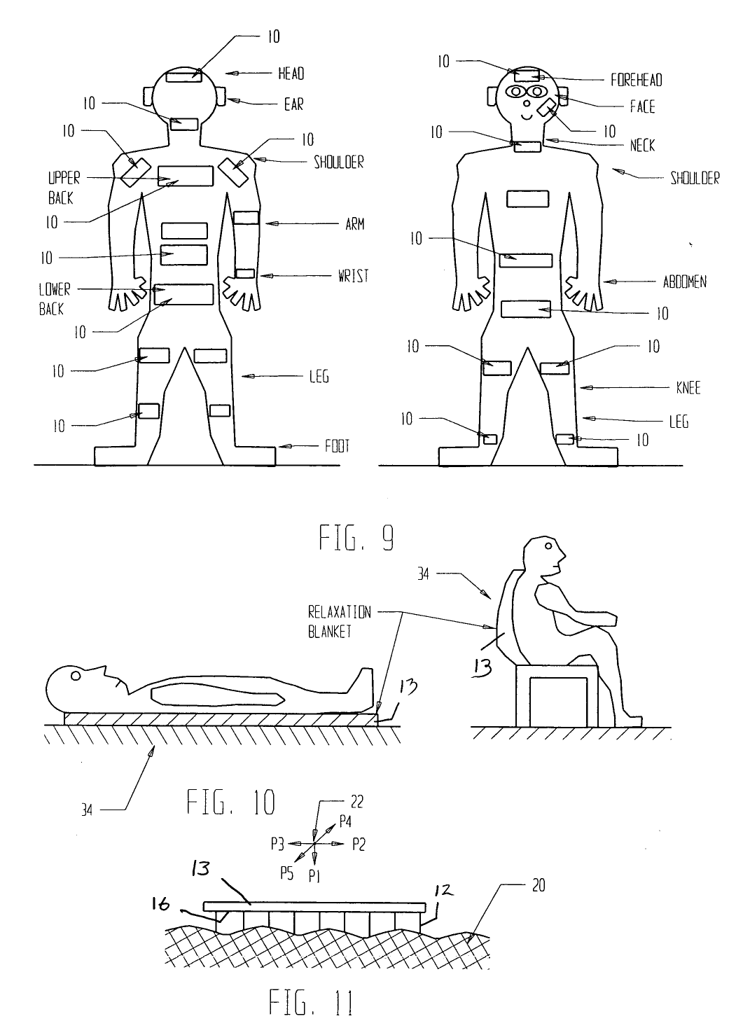

FIG. 9 shows embodiments of the present apparatus 10 applied to different parts of the body. In addition, the apparatus may have the form of a blanket 34, as shown in FIG. 10, which is useful and suggested for athletes relaxing and energizing before, during, and after engaging in athletic activity.

The density of the pins on the therapeutic body must be at least 3 pins per cm² and may be as dense as approximately 1,000 pins per cm². In preferred embodiments, the pin density is between 10 pins per cm² and 200 pins per cm². A density of 100 pins per cm² is suggested. The recommended pin densities as well as the use of pin densities of approximately 1,000 pins per cm² are based on a number of considerations.

The skin above soft tissues is uneven so that the multiple pins 12 are able to contact the skin surface at varying orientations with respect to the skin surface, as shown in FIG. 11. A force is imparted to the skin 20 by the distal end 22 of each pin 12. Because the pins 12 contact the skin surface at varying orientations with respect to the skin surface and because of the flexure of the skin, the force imparted to the skin has a radial component and a normal component. The resultant force will have varying orientations throughout the skin surface which will cause stretching of the superficial skin layer which causes the skin pores to open so as to enhance the absorption of oxygen by the skin.

Although specific embodiments and methods of use have been illustrated and described herein, it will be appreciated by those of ordinary skill in the art that any arrangement that is calculated to achieve the same purpose may be substituted for the specific embodiments and methods shown. It is to be understood that the above description is intended to be illustrative and not restrictive. Combinations of the above embodiments and other embodiments as well as combinations of the above methods of use and other methods of use will be apparent to those having skill in the art upon review of the present disclosure. The scope of the present invention should be determined with reference to the appended claims, along with the full scope of equivalents to which such claims are entitled.

I claim:

1. An apparatus for providing acupressure therapy, comprising:
a therapeutic body having a therapeutic surface; and
a plurality of pins having distal ends and a proximal ends, the proximal ends of the plurality of pins secured to the therapeutic body, the distal end of the pins configured to provide acupressure therapy and extending from the therapeutic surface of the therapeutic body.

2. The apparatus of claim 1, wherein the distal ends of the plurality of pins are sharp.

3. The apparatus of claim 1, wherein the distal ends of the plurality of pins are rounded.

4. The apparatus of claim 1, wherein the plurality of pins are affixed to the therapeutic body in a linear pattern.

5. The apparatus of claim 1, wherein the plurality of pins are affixed to the therapeutic body in a sinusoidal pattern.

6. The apparatus of claim 1, wherein the plurality of pins are affixed to the therapeutic body in a triangular pattern.

7. The apparatus of claim 1, wherein the plurality of pins are affixed to the therapeutic body in a rectangular pattern.

8. The apparatus of claim 1, wherein the plurality of pins are affixed to the therapeutic body in a spiral pattern.

9. The apparatus of claim 1, wherein the therapeutic body includes a cartridge and the cartridge is removably secured within a cartridge holder.

10. The device of claim 1, wherein the plurality of pins are composed of steel.

11. The device of claim 1, wherein the plurality of pins are composed of a plastic.

12. The device of claim 1, wherein the plurality of pins are composed of a precious metal.

13. The device of claim 1, wherein the density of the plurality of pins on the therapeutic body is between 3 pins/1 cm² to 1000 pins/1 cm².

14. The device of claim 1 wherein the therapeutic surface is even.

15. The device of claim 1 wherein the therapeutic surface is uneven.

16. The device of claim 1 wherein the therapeutic body is rigid.

17. The device of claim 1 wherein the therapeutic body is compliant.

18. The device of claim 1 wherein the therapeutic body further comprises a bladder containing a fluid.

19. The device of claim 18 wherein thermal energy is transferred between the bladder and the pins.

20. The device of claim 1 wherein the distal ends of the plurality of pins are connected to an energy source selected from the group consisting of ultrasound, electrical, and laser.
